(19) United States
(12) Patent Application Publication
(10) Pub. No.: US 2002/0006642 A1
(43) Pub. Date: Jan. 17, 2002

(54) METHOD FOR PREPARING A BETA-LACTAM ANTIBIOTIC

(76) Inventors: THOMAS VAN DER DOES, DELFT (NL); JAGDISH CHANDER KAPUR, DELFT (NL); ERIK DE VROOM, LEIDEN (NL)

Correspondence Address: Morrison & Foerster LLP, 3811 Valley Centre Drive, Suite 500, San Diego, CA, 92130-2332, US

(21) Appl. No.: 09/202,799

(22) PCT Filed: Apr. 22, 1998
(86) PCT No.: PCT/EP98/02458

(30) Foreign Application Priority Data
Apr. 22, 1997 (EP) 97201198.5

(51) Int. Cl. C12P 37/04; C12P 21/04

(52) U.S. Cl. 435/71.3; 435/45

(57) ABSTRACT

The invention relates to a method for preparing a β-lactam antibiotic, wherein an N-substituted β-lactam, having general formula (I), wherein R₀ is hydrogen or C₁₋₃ alkoxy; Y is CH₂, oxygen, sulfur, or an oxidized form of sulfur; Z is (a), (b), (c) or (d), wherein R₁ is hydrogen, hydroxy, halogen, C₁₋₃ alkoxy, optionally substituted, optionally containing one or more heteroatoms, saturated or unsaturated, branched or straight C₁₋₅ alkyl, preferably methyl, optionally substituted, optionally containing one or more heteroatoms C₅₋₈ cycloalkyl, optionally substituted aryl or heteroaryl, or optionally substituted benzyl; and X is (CH₂)ₘ-A-(CH₂)ₙ, wherein m and n are the same or different and are chosen from the group of integers 0, 1, 2, 3 or 4 and A is CH═CH, C═C, CHB, C═O, optionally substituted nitrogen, oxygen, sulfur or an optionally oxidized form of sulfur, and B is hydrogen, halogen, hydroxy, C₁₋₃ alkoxy, or optionally substituted methyl, or a salt thereof, is contacted with at least one dicarboxylate acylase, or a functional equivalent thereof, and reacted with a precursor for a side chain of the β-lactam antibiotic in the presence of at least one penicillin acylase, or a functional equivalent thereof.

### FIELD AND BACKGROUND OF THE INVENTION

The invention relates to a method for preparing a β-lactam antibiotic.

The class of β-lactam antibiotics, such as penicillin and cephalosporin antibiotics comprises a great variety of compounds, all having their own activity profile. In general, β-lactam antibiotics consist of a nucleus, the so-called β-lactam nucleus, which is linked through its primary amino group to the so-called side chain via a linear amide bond.

β-Lactam nuclei are very important intermediates in the preparation of semi-synthetic penicillin and cephalosporin antibiotics. The routes to prepare these semi-synthetic penicillins and cephalosporins mostly start from fermentation products such as penicillin G, penicillin V and Cephalosporin C, which are converted to the corresponding β-lactam nuclei, for instance in a manner as is disclosed in K. Matsumoto, Bioprocess. Techn., 16, (1993), 67-88, J. G. Shewale & H. Sivaraman, Process Biochemistry, August 1989, 146-154, T. A. Savidge, Biotechnology of Industrial Antibiotics (Ed. E. J. Vandamme) Marcel Dekker, New York, 1984, or J. G. Shewale et al., Process Biochemistry International, June 1990, 97-103.

Examples of β-lactam nuclei which are employed as precursor for several antibiotics are 6-aminopenicillanic acid (6-APA), 7-aminocephalosporanic acid (7-ACA), 3-chloro-7-aminodesacetoxydesmethylcephalosporanic acid (7-ACCA), 7-aminodesacetylcephalosporanic acid (7-ADAC), and 7-aminodesacetoxycephalosporanic acid (7-ADCA).

The β-lactam nuclei are converted to the desired antibiotic by coupling to a suitable side chain, as has been described in inter alia EP 0 339 751, JP 53005185 and CH 640 240. By making different combinations of side chains and β-lactam nuclei, a variety of penicillin and cephalosporin antibiotics may be obtained, all having their own activity profiles.

For example, D-(−)-phenylglycine, or a suitable derivative thereof, such as an amide or ester, may be attached to any of 7-ACA, 7-ACCA, 7-ADCA and 6-APA to produce Cephaloglycin, Cefaclor, Cephalexin or Ampicillin respectively. Other examples of often employed side chains are D-(−)-4-hydroxyphenylglycine, 2-cyanoacetic acid and 2-(2-amino-4-thiazolyl)-2-methoxyiminoacetic acid.

The known enzymatic methods for preparing β-lactam antibiotics all involve the preparation of a β-lactam nucleus and the subsequent coupling thereof to a suitable side chain. References for enzymatic synthesis are: T. A. Savidge, Biotechnology of Industrial Antiobiotics (Ed. E. J. Vandamme) Marcel Dekker, New York 1984, J. G. Shewale et al., Process Biochemistry International, June 1990 97-103, E. J. Vandamme, Advances in Applied Microbiology, 21, (1977), 89-123 and E. J. Vandamme, Enzyme Microb. Technol., 5, (1983), 403-416. In addition, new routes have been disclosed, which show the direct fermentative production of 7-ADCA and 7-ACA, in EP 0 532 341, EP 0 540 210, WO 93/08287, WO 95/04148 and WO 95/04149.

A disadvantage of these methods is that the coupling reaction of the side chain starts from a β-lactam nucleus, which has to be isolated prior to the coupling reaction. In the isolation of a β-lactam nucleus, which is usually performed by crystallization, up to about 10% of the theoretical yield is lost. Due to the amphoteric nature of the β-lactam nucleus, it dissolves readily in aqueous environment at any pH value and a great part of the production of the β-lactam nucleus is lost in the crystallization mother-liquor.

The present invention overcomes the above disadvantage by introducing the side chain in a reaction which starts from a different material than a β-lactam nucleus.

### DESCRIPTION OF THE INVENTION

It is an object of the invention to provide a method for preparing a β-lactam antibiotic, wherein the side chain is introduced in a reaction which starts from a different material than a β-lactam nucleus.

A further object of the invention is to provide a method for preparing a β-lactam antibiotic, which method may suitably be combined with known enzymatic processes starting from fermentation products such as penicillin G or Cephalosporin C.

Another object of the invention is to provide a method for preparing a β-lactam antibiotic, which method is a clean, efficient and economically feasible process, in other words which method does not result in effluent problems or involve expensive chemicals.

It has been found that the requirements of the above objectives can be met in a method for preparing a β-lactam antibiotic, wherein an N-substituted β-lactam, having the general formula (I)

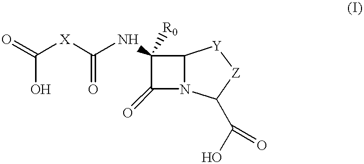

wherein

R₀ is hydrogen or C₁₋₃ alkoxy;

Y is CH₂, oxygen, sulfur, or an oxidized form of sulfur;

Z is

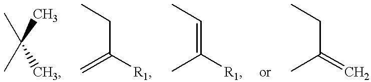

wherein R₁ is hydrogen, hydroxy, halogen, C₁₋₃ alkoxy, optionally substituted, optionally containing one or more heteroatoms, saturated or unsaturated, branched or straight C₁₋₅ alkyl, preferably methyl, optionally substituted, optionally containing one or more heteroatoms C₅₋₈ cycloalkyl, optionally substituted aryl or heteroaryl, or optionally substituted benzyl; and

X is (CH₂)ₘ-A-(CH₂)ₙ, wherein m and n are the same or different and are chosen from the group of integers 0, 1, 2, 3 or 4, and A is CH═CH, C≡C, CHB, C═O, optionally substituted nitrogen, oxygen, sulfur or an optionally oxidized form of sulfur, and B is hydrogen, halogen, hydroxy, C₁₋₃ alkoxy, or optionally substituted methyl,

or a salt thereof, is contacted with at least one dicarboxylate acylase, or a functional equivalent thereof, and reacted with a precursor for a side chain of the β-lactam antibiotic in the presence of at least one penicillin acylase, or a functional equivalent thereof.

Surprisingly, it has been found that β-lactam antibiotics may efficiently be prepared by introducing the side chain of the β-lactam antibiotic in a reaction which starts from an N-substituted β-lactam and wherein two enzymes having different substrates are used. In the process of the invention, it is not necessary to recover the intermediate products, i.e. the product of the first enzymatic reaction, before applying the second enzyme.

Because N-substituted β-lactams may also be prepared from fermentation products, such as penicillin G, penicillin V, cephalosporin C, adipyl-7-ADCA, 3-carboxyethylthiopropionyl-7-ADCA, 2-carboxylethylthioacetyl-7-ADCA, 3-carboxyethylthiopropionyl-7-ADCA, adipyl-7-ACA, 3-carboxyethylthiopropionyl-7-ACA, 2-carboxylethylthioacetyl-7-ACA and 3-carboxyethylthiopropionyl-7-ACA, a great advantage of the invention resides therein that it is now possible to enzymatically prepare β-lactam antibiotics, starting from such fermentation products, without the isolation of a β-lactam nucleus intermediate, which isolation causes a significant loss of product.

A method according to the invention is a clean and highly specific process. This means, that no or hardly no by-products are generated which would cause effluent and/or purification problems. Furthermore, a method according to the invention does not require the use of complex and expensive reagents, which are usually difficult to handle due to their sensitivity.

Suprisingly, it has been found that no significant enzyme inhibition effect occurs in a method according to the invention. Up until now, it has been believed that transacylation using one or two enzymes in the preparation of β-lactam antibiotics is not possible due to an enzyme inhibition effect. It was expected that in the transacylation reaction phenylacetic acid or phenoxyacetic acid would be formed, which acids act as inhibitors for certain enzymes as has been reported by U. Schömer et al., Applied and Environment Microbiology, (February 1984), 307-312 and by A. L. Margolin et al. in Biochim. Biophys. Acta, 616, (1980), 283-289.

The starting material in a method according to the invention is an N-substituted β-lactam having the above general formula (I) or a salt thereof. In the above definitions of the various symbols in formula (I), an oxidized form of sulfur is meant to include groups such as sulfoxide and sulfone. By optionally substituted alkyl, cycloalkyl, aryl, heteroaryl and benzyl, groups are intended, which have substituents such as alkyl groups of from 1 to 3 carbon atoms. Optionally substituted nitrogen includes primary, secondary and tertiary amine groups, which may be substituted with for instance alkyl groups of from 1 to 3 carbon atoms. Optionally substituted methyl is meant to include a methyl group and various substituted methyl groups such as —CHₚD_{q}, wherein D is a halogen and p and q are integers of which the sum equals 3.

Formula (I) is intended to encompass N-substituted β-lactams, which are based on any β-lactam nucleus disclosed in “Cephalosporins and Penicillins, Chemistry and Biology”, Ed. E. H. Flynn, Academic Press, 1972, pages 151-166, and “The Organic Chemistry of β-Lactams”, Ed. G. I. Georg, VCH, 1992, pages 89-96, which are incorporated herein by reference. Preferred are those starting materials wherein R₁ represents a CH₂-E or CH═CH-E group, wherein E is hydrogen, hydroxy, halogen, C₁₋₃ alkoxy, optionally substituted, optionally containing one or more heteroatoms, saturated or unsaturated, branched or straight C₁₋₅ alkyl, optionally substituted, optionally containing one or more heteroatoms C₅₋₈ cycloalkyl, optionally substituted aryl or heteroaryl, or optionally substituted benzyl.

Suitable salts of the N-substituted β-lactam starting material include any non-toxic salt, such as an alkali metal salt (e.g. sodium or potassium), an alkali earth metal salt (e.g. calcium or magnesium), an ammonium salt, or an organic base salt (e.g. trimethylamine, triethylamine, pyridine, picoline, dicyclohexylamine, N,N′-dibenzyl diethylene diamine).

The N-substituted β-lactam starting material having general formula (I) may be enzymatically prepared, for instance in a method as disclosed in EP 0 532 341, WO 95/04148 or WO 95/04149. Preferred starting materials are N-glutaryl, N-succinyl, N-adipyl, N-3-(carboxymethylthio)propionyl, N-trans-β-hydromuconyl, N-pimelyl or N-3,3′-thiodipropionyl β-lactam, or salts thereof. Starting materials based on these dicarboxylic acids are efficiently converted by the enzymes used in accordance with the invention.

Further preferred starting materials are N-substituted 6-aminopenicillanic acid (6-APA), N-substituted 7-aminocephalosporanic acid (7-ACA), N-substituted 3-chloro-7-aminodesacetoxydesmethylcephalosporanic acid (7-ACCA), N-substituted 7-aminodesacetylcephalosporanic acid (7-ADAC), or N-substituted 7-aminodesacetoxycephalosporanic acid (7-ADCA), as these N-substituted β-lactams result in β-lactam antibiotics having the most advantageous activity profiles.

A suitable dicarboxylate acylase with which the N-substituted β-lactam is contacted in a method according to the invention is an enzyme that may be isolated from various naturally occurring micro-organisms, such as fungi and bacteria. Such micro-organisms can be screened for enzymes with the desired dicarboxylic acid specificity by monitoring the hydrolysis of suitable substrates. Such suitable substrates may be e.g. chromophores such as succinyl-, glutaryl- or adipyl-p-nitroanilide. Also, the hydrolysis of the corresponding N-substituted β-lactams may be used for identifying the required enzymes. It was found that the optimum pH range for these enzymes lies between about 6, preferably about 7, and about 9, preferably about 8.

Organisms that have been found to produce dicarboxylate acylase are Alcaligenes, Arthrobacter, Achromobacter, Aspergillus, Acinetobacter, Bacillus and Pseudomonas species. More in particular, the following species produce highly suitable dicarboxylate acylases: Achromobacter xylosooxidans, Arthrobacter viscosis, Arthrobacter CA128, Bacillus CA78, Bacillus megaterium ATCC53667, Bacillus cereus, Bacillus laterosporus J1, Paecilomyces C2106, Pseudomonas diminuta sp N176, Pseudomonas diminuta sp V22, Pseudomonas paucimobilis, Pseudomonas diminuta BL072, Pseudomonas strain C427, Pseudomonas sp SE83, Pseudomonas sp SE495, Pseudomonas ovalis ATCC950, Comamonas sp SY77, Pseudomonas GK 16, Pseudomonas SY-77-1, Pseudomonas sp A14, Pseudomonas vesicularis B965, Pseudomonas syringae, Ps putida ATCC17390, Ps aeroginosa NCTC 10701, Proteus vulgaris ATCC9634, Ps fragi DSM3881, and B. subtilus IFO3025.

The dicarboxylate acylase may be obtained from the micro organism by which it is produced in any suitable manner, for example as is described for the Pseudomonas sp SE83 strain in U.S. Pat. No. 4,774,179. Also, the genes for e.g. SE83 or SY77 dicarboxylate acylases may be expressed in a different suitable host, such as E. coli, as has been reported by Matsuda et al. in J. Bacteriology, 169, (1987), 5818-5820 for the SE83 strain, and in U.S. Pat. No. 5,457,032 for the SY77 strain.

The enzymes isolated from the above sources are often referred to as glutaryl acylases. However, the side chain specificity of the enzymes is not limited to the glutaryl side chain, but comprises also smaller and larger dicarboxyl side chains. Some of the dicarboxylate acylases also express gamma-glutamyl transpeptidase activity and are therefore sometimes classified as gamma-glutamyl transpeptidases.

A suitable penicillin acylase with which the N-substituted β-lactam is contacted in a method according to the invention is an enzyme that may be isolated from various naturally occurring micro organisms, such as fungi and bacteria. Such micro organisms can be screened For enzymes with the desired specifity in a monitoring test analogous to the one described for the dicarboxylate acylase. Of these enzymes it was found that the optimum pH lies between about 4, preferably, about 5, and about 7, preferably about 6.

Organisms that have been found to produce penicillin acylase are, for example, Acetobacter, Aeromonas, Alcaligenes, Aphanocladium, Bacillus sp., Cephalosporium, Escherichia, Flavobacterium, Kluyvera, Mycoplana, Protaminobacter, Providentia, Pseudomonas or Xanthomonas species. Enzymes derived from Acetobacter pasteurioanum, Alcaligenes faecalis, Bacillus megaterium, Escherichia coli, Providentia rettgeri and Xanthomonas citrii have particularly proven to be successful in a method according to the invention. In the literature, penicillin acylases have also been referred to as penicillin amidases.

The dicarboxylate acylase and penicillin acylase may be used as free enzymes, but also in any suitable immobilized form, for instance as has been described in EP 0 222 462 and WO 97/04086. It is possible to perform a method according to the invention wherein both enzymes are immobilized on one carrier or wherein the enzymes are immobilized on different carriers. In addition, it is possible to use functional equivalents of one or both of the enzymes, wherein for instance properties of the enzymes, such as pH dependence, thermostability or specific activity may be affected by chemical modification or crosslinking, without significant consequences for the activity, in kind, not in amount, of the enzymes in a method according to the invention. Also, functional equivalents such as mutants or other derivatives, obtained by classic means or via recombinant DNA methodology, biologically active parts or hybrids of the enzymes may be used. In some cases, modification, chemical or otherwise, may be beneficial in a method according to the invention, as is part of the standard knowledge of the person skilled in the art.

The precursor for a side chain of the β-lactam antibiotic to be prepared in a method according to the invention may be any compound that is recognized by the above defined penicillin acylases and leads to a product of the class of β-lactam antibiotics. Preferably, the substrate is chosen from the group of D-(−)-phenylglycine, D-(−)-4-hydroxyphenylglycine, D-(−)-2,5-dihydrophenylglycine, 2-thienylacetic acid, 2-(2-amino-4-thiazolyl)-2-methoxyiminoacetic acid, α-(4-pyridylthio)acetic acid, 3-thiophenemalonic acid, or 2-cyanoacetic acid, and derivatives thereof, as these substrates lead to β-lactam antibiotics having the most advantageous activity profile. Suitable derivatives of these substrates are esters and amides, wherein the side chain molecule is connected to a C₁-C₃ alkyl group through an ester or amide linkage.

In a method according to the invention the dicarboxylate acylase, the precursor for the side chain of the β-lactam antibiotic and the penicillin acylase may be added to the N-substituted β-lactam starting material together or apart. Preferably, the enzymes are added together to the N-substituted β-lactam and the precursor for the side chain.

In a preferred embodiment of the invention, a process is carried out without isolation and/or purification of any intermediates that may at one time or another be present in the reaction mixture. This way, no product is lost in an isolation or purification process.

In a highly preferred embodiment of the invention, a process is carried out as a one-pot process. By “one-pot process” any process is meant wherein the complete process is carried out in one reactor vessel. In other words, essentially no major reaction components are drawn off out of the reactor vessel at any time during the time a method according to the invention is carried out. The advantages of this embodiment will be evident to the skilled person.

The conditions applied in a method according to the invention depend on various parameters, in particular the type of reagents, the concentration of reagents, reaction time, titrant, temperature, pH, enzyme concentration, and enzyme morphology. Given a specific N-substituted β-lactam that is to be converted to a given β-lactam antibiotic using a given dicarboxylate acylase and a given penicillin acylase, the person skilled in the art will be able to suitably choose the optimum reaction conditions.

It has, however, been found that the optimum reaction temperature in a method according to the invention lies between 0 and 80° C., preferably between 10 and 50° C. The optimum pH in the preparation of a β-lactam antibiotic according to the invention lies between 4.5 and 9.0. In this regard, it is to be noted that is highly preferable to perform a method according to the invention in aqueous environment, because thus the use of organic solvents, which would lead to effluent problems, is circumvented. Moreover, both the dicarboxylate acylase and the peniclline acylase enzymes have proven to catalyze the conversion reaction most efficiently in an aqueous environment.

Generally, the reagents will be present in amounts ranging between 0.01, preferably 0.5, and 3 mol per kilogram reaction mixture, preferably 2 mol per kilogram reaction mixture, in both steps.

Suitable enzyme concentrations are chosen such that the total reaction time does not exceed 4 hours. For the conversion of 10 millimole of substrate into product within one hour, about 500 to 3000 enzyme reaction units should be applied, wherein an enzyme reaction unit is defined as the amount of enzyme which converts one micromole of substrate into product in one minute under conditions which represent the actual process conditions. In general, for the conversion of a certain amount of substrate in one hour, the enzyme dosage should preferentially be between 50 and 300 kUnits per mole. However, usually a larger excess of activity is dosed in order to compensate for any losses which may occur during the process.

Suitable titrants are inorganic acids and bases, such as hydrochloric acid, sulfuric acid, nitric acid, phosphoric acid, sodium hydroxide, potassium hydroxide, sodium bicarbonate, potassium bicarbonate, ammonium hydroxide, and so forth, or organic acids, such as formic acid, acetic acid, succinic acid, adipic acid, glutaric acid and so forth. Titrant concentration may vary between 0.01 and 8 M, depending on the scale of the reaction and the solubility of the titrant.

Of course the invention also encompasses a β-lactam antibiotic obtainable by the methods disclosed hereinabove.

The invention will now be elucidated by the following non-restrictive examples.

### EXAMPLES

### Definitions and Procedures

Enzyme Activity

As definition of penicillin G acylase activity the following was used: one unit (U) corresponds to the amount of enzyme that hydrolyses 1 micromole penicillin G per minute under standard conditions (100 g.1-1 penicillin G potassium salt, 0.05 M potassium phosphate buffer, pH 8.0, 28° C.).

As definition of dicarboxylate acylase activity the following was used: one unit (U) corresponds to the amount of enzyme that hydrolyses 1 mmol N-adipyl-7-ADCA per minute under standard conditions (100 mM N-adipyl-7-ADCA, 100 mM Tris buffer, pH 8.0, 37° C.).

pH Measurement

A Mettler DL21 titration apparatus equipped with an automatic burette and a Brother M1509 printing device was used.

| HPLC analysis | |
|---|---|
| For Amoxicillin: | |
| Column: | Chromsphere C18, 5 Mm (100 × 3.0 nm) |
| Solvent: | 25% acetonitrile in 12 mM potassium |
| | phosphate buffer containing 0.2 % sodium |
| | dodecyl sulphate |
| Flow: | 1 ml.min⁻¹ |
| Detection: | 214 nm |
| For Cephalexin: | |
| Column: | Micromsphere C18, 3 Mm (100 × 4.6 mm) |
| Solvent: | 29% acetonitrile in 14 mM potassium |
| | dihydrogen phosphate buffer, pH 3.0 with |
| | phosphoric acid |
| Flow: | 1 ml.min⁻¹ |
| Detection: | 254 nm |

### Example 1

### Amoxicillin from N-adipyl-6β-aminopenicillanic Acid and D-(−)-4-hydroxyphenylglycine Methyl Ester

To a solution of dipotassium N-adipyl-6β-aminopenicillinate (0.71 g, purity 59%; 1.0 mmol) and D-(−)-4-hydroxyphenylglycine methyl ester (0.45 g, purity >97%, 2.4 mmol) in water (10 ml) was added dicarboxylate acylase obtained from Pseudomonas SE83 (1.044 g, 96 U.g⁻¹) and penicillin acylase obtained from Escherichia coli (0.80 g, 125 U.g¹). The mixture was stirred at room temperature and the pH was maintained at 6.9 using a 1M solution of sodium hydroxide in water. Formation of products was monitored using HPLC analysis. The results are shown in Table 1.

TABLE 1

| Time (h) | Adipyl-6-APA (mM) | 6-APA (mM) | Amoxicillin (mM) |
|---|---|---|---|
| 0 | 121 | 0 | 0 |
| 0.5 | 81 | 13 | 4 |
| 1.0 | 80 | 12 | 7 |

### Example 2

### Cephalexin from N-adipyl-7-amino-3-methylceph-3-em-4-carboxylate and D-(−)-phenylglycine Amide

To a solution of N-adipyl-7-amino-3-methylceph-3-em-4-carboxylate (0.68 g, purity 97.1%, 2.0 mmol) and D-(−)-phenylglycine amide (0.75 g, purity 96%, 4.8 mmol) in water (20 ml) dicarboxylate acylase obtained from Pseudomonas SE83 (4.00 g, 369 U.g⁻¹) and penicillin acylase obtained from Escherichia coli (1.6 g, 250 U.g⁻¹) were added. The starting pH of the reaction mixture was 6.3, the reaction mixture was stirred at 35° C., and after 30 minutes (pH=6.8) HPLC analysis showed the formation of Cephalexin. For HPLC analysis, 0.5 ml of the reaction mixture was taken out of the reaction vessel, centrifuged and from the filtrate, a volume of 0.2 ml was made up to 50 ml with buffer solution of pH 7. The results are shown in Table 2.

TABLE 2

| Time (h) | Adipyl-7-ADCA (mM) | 7-ADCA (mM) | Cephalexin (mM) |
|---|---|---|---|
| 0 | 95 | 0 | 0 |
| 0.5 | 34 | 35 | 9.9 |

### Example 3

### Cephalexin from N-adipyl-7-amino-3-methylceph-3-em-4-carboxylate and D-(−)-phenylglycine Amide

To a solution of N-adipyl-7-amino-3-methylceph-3-em-4-carboxylate (0.68 g, purity 97.1%, 2,0 mmol) in water (20 ml) dicarboxylate acylase obtained from Pseudomonas SE83 (4.00 g, 369 U.g⁻¹) was added. The reaction mixture was stirred at 35° C. and the pH was maintained at 8.0 by using a 2 M solution of potassium hydroxide in water. After about one hour, the reaction contents were filtered and to the combined filtrate D-(−)-phenylglycine amide (0.75 g, purity 96%, 4.8 mmol) and penicillin acylase obtained from Escherichia coil (1.6 g, 250 U.g⁻¹) were added. The reaction contents were maintained at 13° C. and pH 7.5 by using a 1 M solution of hydrochloride in water. HPLC analysis showed the formation of cephalexin. For HPLC analysis, 0.5 ml of the reaction mixture was taken out of the reaction vessel, centrifuged and from the filtrate, a volume of 0.2 ml was made up to 25 ml with buffer solution of pH 7. The results are shown in Table 3.

TABLE 3

| Time (h) | Adipyl-7-ADCA (mM) | 7-ADCA (mM) | Cephalexin (mM) |
|---|---|---|---|
| 0 | 95 | 0 | 0 |
| 1.0 | 2.2 | 32 | 0 |
| 2.0 | 3.7 | 14 | 37 |

1. A method for preparing a β-lactam antibiotic, wherein an N-substituted β-lactam, having the general formula (I) 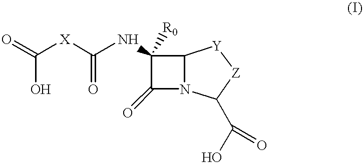
wherein
R₀ is hydrogen or C₁₋₃ alkoxy;
Y is CH₂, oxygen, sulfur, or an oxidized form of sulfur;
Z is 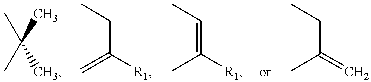
wherein R₁ is hydrogen, hydroxy, halogen, C₁₋₃ alkoxy, optionally substituted, optionally containing one or more heteroatoms, saturated or unsaturated, branched or straight C₁₋₅ alkyl, preferably methyl, optionally substituted, optionally containing one or more heteroatoms C₅₋₈ cycloalkyl, optionally substituted aryl or heteroaryl, or optionally substituted benzyl; and
X is (CH₂)ₘ-A-(CH₂)ₙ, wherein m and n are the same or different and are chosen from the group of integers 0, 1, 2, 3 or 4, and A is CH═CH, C≡C, CHB, C═O, optionally substituted nitrogen, oxygen, sulfur or an optionally oxidized form of sulfur, and B is hydrogen, halogen, hydroxy, C₁₋₃ alkoxy, or optionally substituted methyl,
or a salt thereof, is contacted with at least one dicarboxylate acylase, or a functional equivalent thereof, and reacted with a precursor for a side chain of the β-lactam antibiotic in the presence of at least one penicillin acylase, or a functional equivalent thereof.

2. A method according to claim 1, wherein no intermediate products are isolated and/or purified.

3. A method according to claim 2, which is performed as a one-pot process.

4. A method according to any of the preceding claims, wherein the N-substituted β-lactam is an N-glutaryl, N-succinyl, N-adipyl, N-3-(carboxymethylthio)propionyl, N-trans-β-hydromuconyl, N-pimelyl or N-3,3′-thiodipropionyl β-lactam, or a salt thereof.

5. A method according to any of the preceding claims, wherein the N-substituted β-lactam is an N-substituted 6-aminopenicillanic acid (6-APA), 7-aminocephalosporanic acid (7-ACA), 3-chloro-7-aminodesacetoxydesmethylcephalosporanic acid (7-ACCA), 7-aminodesacetylcephalosporanic acid (7-ADAC), or 7-aminodesacetoxycephalosporanic acid (7-ADCA), or a salt thereof.

6. A method according to any of the preceding claims, wherein the precursor for a side chain of the β-lactam antiobiotic is D-(−)-phenylglycine, D-(−)-4-hydroxyphenylglycine, D-(−)-2,5-dihydrophenylglycine, 2-thienylacetic acid, 2-(2-amino-4-thiazolyl)-2-methoxyiminoacetic acid, α-(4-pyridylthio)acetic acid, 3-thiophenemalonic acid, or 2-cyanoacetic acid, or an amide or ester thereof.

7. A method according to any of the preceding claims, wherein the dicarboxylate acylase is obtained from an Alcaligenes, Arthrobacter, Achromobacter, Aspergillus, Acinetobacter, Bacillus or a Pseudomonas species.

8. A method according to any of the preceding claims, wherein the penicillin acylase is obtained from an Acetobacter, Aeromonas, Alcaligenes, Aphanocladium, Bacillus sp., Cephalosporium, Escherichia, Flavobacterium, Kluyvera, Mycoplana, Protaminobacter, Providentia, Pseudomonas or a Xanthomonas species.

9. A method according to any of the preceding claims, wherein the N-substituted β-lactam is obtained by an enzymatic process starting from a fermentation product.

10. A method according to claim 9, wherein the fermentation product is penicillin G, penicillin V, Cephalosporin C, adipyl-7-ADCA, 3-carboxyethylthiopropionyl-7-ADCA, 2-carboxylethylthioacetyl-7-ADCA and 3-carboxyethylthiopropionyl-7-ADCA, adipyl-7-ACA, 3-carboxyethylthiopropionyl-7-ACA, 2-carboxylethylthioacetyl-7-ACA and 3-carboxyethylthiopropionyl-⁷-ACA.

11. Use of a dicarboxylate acylase and a penicillin acylase to convert an N-substituted β-lactam to a β-lactam antibiotic.
